(19) United States
(12) Patent Application Publication
(10) Pub. No.: US 2013/0312739 A1
(43) Pub. Date: Nov. 28, 2013

(54) Aerosol Generator

(71) Applicants: Mark Rome, London (GB); Tom Woodman, London (GB)

(72) Inventors: Mark Rome, London (GB); Tom Woodman, London (GB)

(73) Assignee: BRITISH AMERICAN TOBACCO (INVESTMENTS) LIMITED, London (GB)

(21) Appl. No.: 13/825,477

(22) PCT Filed: Sep. 30, 2011
(86) PCT No.: PCT/EP2011/067148

§ 371 Date: Aug. 14, 2013

(30) Foreign Application Priority Data
Oct. 6, 2010 (GB) 1016797.1

(51) Int. Cl. A24F 47/00; A61M 11/02

(52) U.S. Cl. CPC A24F 47/002; A61M 11/02
USPC 128/200.22

(57) ABSTRACT

An aerosol generator (19), comprising a storage chamber (10), a dosing chamber (20) and an expansion chamber (30), wherein the storage chamber (10), dosing chamber (20) and expansion chamber (30) reside in a single unit and wherein the dosing chamber (20) defines a maximum volume of fluid that may be delivered from the storage chamber (10) to the expansion chamber (30), is disclosed.

The present invention relates to an aerosol generator device.

Recently, conventional smoking articles, which involve combustion of smokable material, have been replaced with aerosol generators which can dispense pressurised nicotine containing fluids. Aerosol generators provide a means for users to receive a substance such as nicotine in a manner which closely resembles conventional smoking but without combusting smokable material.

Hitherto, aerosol generators have contained a quantity of pressurised fluid, such as a fluid containing a propellant and nicotine, corresponding to a single smoking article. Although the aerosol generator may be refilled, as described in WO 2009/001082, this approach requires the device to dock with a separate refilling unit. Such an arrangement is unnecessarily cumbersome and inconvenient.

The present invention provides an aerosol generator comprising a storage chamber; a dosing chamber; and an expansion chamber, wherein the storage chamber, dosing chamber and expansion chamber reside in a single unit and wherein the dosing chamber defines a maximum volume of fluid that may be delivered from the storage chamber to the expansion chamber.

In order that the present invention may be more fully understood an embodiment thereof will be described with reference to the accompanying drawings of which:

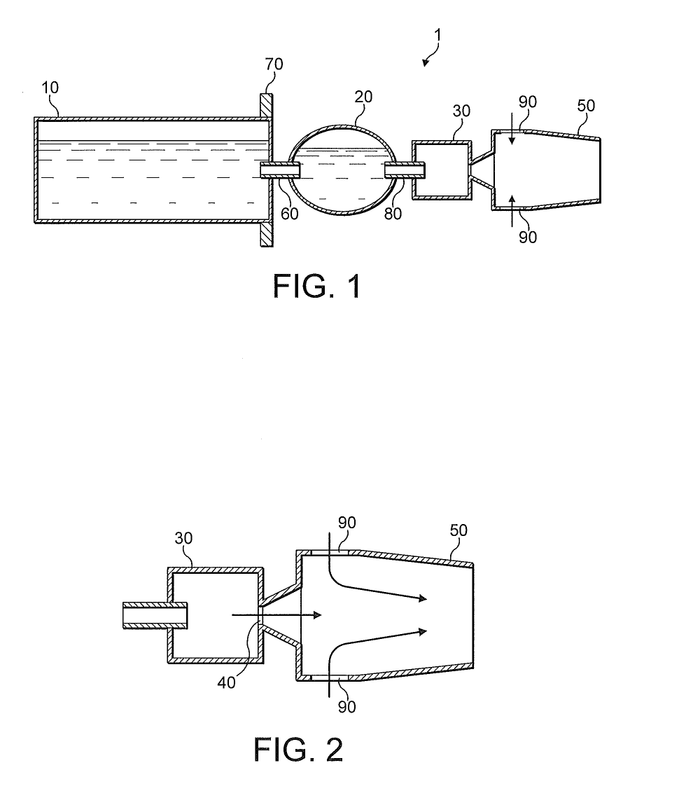

FIG. 1 is a side view of an aerosol generator; and

FIG. 2 is an exploded side view of part of the aerosol generator of FIG. 1.

FIG. 1 shows an aerosol generator 1 according to one embodiment of the present invention. The aerosol generator 1 comprises a storage chamber 10 such as a canister, a dosing chamber 20, an expansion chamber 30 and an orifice 40 (shown in FIG. 2) separating the expansion chamber 30 from a mouthpiece 50. The aerosol generator 1 is an integral unit and may be housed in a single casing (not shown). Alternatively, the aerosol generator 1 may be formed by joining the storage chamber 10, dosing chamber 20, expansion chamber 30 and mouthpiece 50 together without employing an outer casing.

The storage chamber 10 can hold a predetermined volume of pressurised fluid such as a nicotine containing substance or mixture. The storage chamber 10 may be pressurised with a gas such as nitrogen or carbon dioxide or any other suitable gas that would be obvious to the person skilled in the art. The storage chamber 10 may contain a propellant such as hydrofluoroalkane however other propellants that are known in the art may be employed.

The storage chamber 10 is separated from the dosing chamber 20 by a valve 60. The valve 60 may be a continuous flow valve and constitutes part of a priming mechanism which regulates the flow of pressurised fluid from the storage chamber 10 to the dosing chamber 20. The valve 60 may alternatively be a metered or unmetered valve or any other suitable valve known in the art. The valve 60 may be opened by pressing a button 70 which is located on the storage chamber 10 or on any other suitable part of the aerosol generator 1. Alternative means of priming the dosing chamber 20 that are known in the art may be employed for example by substituting a lever in place of the button 70 or by twisting the storage chamber 10 relative to the rest of the aerosol generator 1 to open the valve 60.

Once the valve 60 is opened pressurised fluid is released from the storage chamber 10 into the dosing chamber 20. The fluid flows into the dosing chamber 20 until the pressure inside the dosing chamber 20 is equal to the pressure in the storage chamber 10. When the pressure has equalised the net flow of fluid from the storage chamber 10 into the dosing chamber 20 will cease and the valve 60 may be closed.

The storage chamber 10 may be dimensioned to contain a volume of pressurised fluid equivalent to a predetermined number of cigarettes or other smoking articles. The dosing chamber 20 may be dimensioned to contain a volume of pressurised fluid equivalent to a predetermined number of puffs or draws of a cigarette or other smoking article. Such a predetermined number of puffs may correspond to the average number of puffs obtained from a single smoking article. As such, an aerosol generator 1 according to the present invention possesses the advantage of isolating a quantity of pressurised fluid that may be equivalent to a single smoking article from a quantity of pressurised fluid that may be equivalent to several smoking articles or a pack of smoking articles. Alternatively, a volume of pressurised fluid corresponding to one puff of a smoking article may be isolated, from the storage chamber 10, in the dosing chamber 20. A user may then obtain the pressurised fluid containing nicotine or other pressurised fluid contained in the dosing chamber 20 in a manner which will now be described.

Once the dosing chamber 20 has been primed and the valve 60 closed, the user can draw the pressurised fluid from the dosing chamber 20 into the expansion chamber 30 through the mouthpiece 50 and actuating a valve 80 located between the dosing chamber 20 and the expansion chamber 30.

The valve 80 shown in FIG. 1 may be puff actuated however alternative valves such as metered or unmetered valves may also be used.

Inside the expansion chamber 30 the pressurised fluid and propellant expand and are directed through the orifice 40 to form an aerosol. Inside the expansion chamber 30 some of the liquid propellant changes phase from a liquid to a gas which causes an increase in pressure to force the aerosol through the orifice 40 into the mouthpiece 50.

As can be seen in FIG. 2, the mouthpiece 50 comprises ventilation holes 90 so that when the user draws through the mouthpiece 50 ambient air at atmospheric pressure is drawn through the holes 90 due to the creation of a pressure differential with the pressure in the mouthpiece 50 lower than the pressure of ambient air. The ventilation holes 90 provide dilution of the aerosol as well as providing the pressure differential between the mouthpiece 50 and the ambient air. In the case where the valve 80 is puff actuated this feature can lead to the valve 80 opening, thereby releasing the mixture of pressurised fluid and propellant from the dosing chamber 20 into the expansion chamber 30.

The particle size of the aerosol thus generated is dependent on the volume of pressurised fluid and propellant delivered to the expansion chamber 30, the size of the orifice 40 and the size of the expansion chamber 30. The volume of the aerosol will depend on the duration of each puff taken by the user or on the size of the valve 80 if the valve 80 is a metered valve.

The arrows in FIG. 2 represent the flow of aerosol through the orifice 40 and the flow of air through the ventilation holes 90. The flow rate of aerosol generated in the expansion chamber 30 between the orifice 40 and mouthpiece 50 is dependent on the size of the orifice 40.

The flow rate of ambient air through the ventilation holes 90 is dependent on the size of the ventilation holes 90 and they may be dimensioned to achieve a desired flow rate similar to the flow rate obtained when smoking a cigarette or other smoking article well known in the art. For example, a flow rate of approximately 27 millilitres per second (ml/s) through the ventilation holes 90 is known to correspond to intense smoking conditions. Inside the mouthpiece 50 the aerosol mixes with ambient air that has entered via the ventilation holes 90 to produce the desired puff volume.

After the user has ceased drawing the aerosol through the mouthpiece 50, the valve 80 may be closed. In the case where the valve 80 is puff actuated this will be due to the fact that the pressure differential which caused the valve 80 to open is no longer present and a spring or other such biasing device (not shown) may close the valve. If the valve 80 is not puff actuated it may be closed manually.

The aerosol may be drawn by the user until the pressurised fluid inside the dosing chamber 20 has been exhausted. The dosing chamber 20 may be topped up or refilled by actuating the valve 60 to release pressurised fluid from the storage chamber 10 into the dosing chamber 20. Once the contents of the storage chamber 10 have been exhausted, the storage chamber may be removed and replaced with a storage chamber 10 containing pressurised fluid.

The embodiment described in the foregoing description is intended merely as an illustrative example of the present invention. Modifications may be made without departing from the scope of the invention which is defined by the claims listed hereinafter.

1. An aerosol generator comprising:
a storage chamber;
a dosing chamber; and
an expansion chamber,
wherein the storage chamber, dosing chamber and expansion chamber reside in a single unit and
wherein the dosing chamber defines a maximum volume of a fluid deliverable from the storage chamber to the expansion chamber.

2. The aerosol generator according to claim 1,
wherein the storage chamber and the dosing chamber are separated by a continuous flow valve.

3. The aerosol generator according to claim 1,
wherein the dosing chamber and the expansion chamber are separated by a puff actuated valve.

4. The aerosol generator according to claim 1, further comprising a mouthpiece.

5. The aerosol generator according to claim 4
further comprising an orifice between the expansion chamber and the mouthpiece.

6. The aerosol generator according to claim 4,
wherein the mouthpiece comprises at least one ventilation hole.

7. The aerosol generator according to claim 6,
wherein the at least one ventilation hole and mouthpiece are configured to deliver an aerosol at a predetermined flow rate.

8. The aerosol generator according to claim 7, wherein the predetermined flow rate is approximately 27 millilitres per second.

9. The aerosol generator according to claim 1,
wherein the fluid contains nicotine.

10. The aerosol generator according to claim 9,
wherein the volume of fluid containing nicotine that can be held in the dosing chamber corresponds to a quantity of nicotine contained in a smoking article.

11. The aerosol generator according to claim 9,
wherein the volume of fluid containing nicotine that can be held in the dosing chamber corresponds to a quantity of nicotine contained in a predetermined number of puffs of a smoking article.

12. (canceled)

13. An aerosol generator comprising:
a fluid storage chamber;
a dosing chamber;
a mouthpiece;
an expansion chamber; and
an orifice between the expansion chamber and the mouthpiece,
wherein the fluid storage chamber, dosing chamber and expansion chamber form a single unit without employing an outer casing; and
wherein the dosing chamber defines a maximum volume of a fluid deliverable from the fluid storage chamber to the expansion chamber after a single priming event.

14. The aerosol generator according to claim 13,
wherein the fluid storage chamber and the dosing chamber are separated by one of a continuous flow valve and a puff actuated valve.

15. The aerosol generator according to claim 13,
wherein the orifice and a ventilation hole are configured to deliver an aerosol at a predetermined flow rate.

16. The aerosol generator according to claim 13,
wherein the fluid is a nicotine-containing fluid.

17. An aerosol generator comprising:
a replaceable fluid storage chamber;
a dosing chamber;
a mouthpiece;
an expansion chamber; and
an orifice between the expansion chamber and the mouthpiece,
wherein the fluid storage chamber is removable; and
wherein the dosing chamber defines a maximum volume of fluid that may be delivered from the fluid storage chamber to the expansion chamber after a single priming event.

18. The aerosol generator according to claim 17,
wherein the fluid storage chamber and the dosing chamber are separated by one of a continuous flow valve and a puff actuated valve.

19. The aerosol generator according to claim 17,
wherein the orifice and a ventilation hole are configured to deliver an aerosol at a predetermined flow rate.

20. The aerosol generator according to claim 17,
wherein the fluid is a nicotine-containing fluid.

21. The aerosol generator according to claim 20,
wherein the maximum nicotine-containing fluid capacity of the dosing chamber corresponds to a quantity of nicotine contained in a predetermined number of puffs of a smoking article.
